(19) United States
(12) Patent Application Publication
(10) Pub. No.: US 2023/0344188 A1
(43) Pub. Date: Oct. 26, 2023

(54) METHOD TO GENERATE COHERENT ULTRAVIOLET RADIATION FROM LASER BEAMS

(71) Applicant: UVL A/S, Åbyhøj (DK)

(72) Inventors: Emil Zanchetta Ulsig, Aarhus V (DK); Nicolas Volet, Risskov (DK); Peter Johansen, Allingabro (DK)

(21) Appl. No.: 18/026,976

(22) PCT Filed: Sep. 18, 2021
(86) PCT No.: PCT/IB2021/058521

§ 371 Date: Mar. 17, 2023

(30) Foreign Application Priority Data
Sep. 20, 2020 (IN) 202041040682

(51) Int. Cl. H01S 3/109; H01S 3/0941; H01S 3/094

(52) U.S. Cl. CPC H01S 3/109; H01S 3/0941; H01S 3/094096

(57) ABSTRACT

A laser source device including a pump laser system, a combinator, and nonlinear frequency generation module. The pump laser system is made of one of more laser diodes, each of which generates one or more light beams whose wavelengths are in the visible spectral range having a wavelength in the range 380 nm to 740 nm. The combinator allows passing of the light beams from the pump laser system and combines them and couples them to the next module. The nonlinear frequency generation module allows passing of the light beams and, while passing through, employs nonlinear optical phenomenon of sum-frequency generation for providing a UVC light beam.

### FIELD OF INVENTION

The invention is related to the generation of coherent ultraviolet radiation. More specifically, the invention is related to the conversion of laser light in the ultraviolet C-band from laser beams in the visible spectrum.

### BACKGROUND OF THE INVENTION

There is an expanding need for non-contagious environments and alternative effective germicidal sterilization methods. Light in the ultraviolet C-band (UVC) has proven effective in sterilizing air and surfaces, while also being safe for humans and other mammals.

It has been established that light between 207 nm and 230 nm cannot penetrate even the outer (non-living) layers of human skin or eye, however, because bacteria and viruses are of micrometer or smaller dimensions, far-UVC can penetrate and inactivate them. As a result, 222-nm light efficiently inactivates bacteria without harm to exposed mammalian skin. In addition, proteins have a strong absorption maximum around 220 nm. As a result, for inactivation purposes, this wavelength is the most effective. Furthermore, penetration of −220-nm light through the cornea to the lens is predicted to be essentially zero. This suggests that 220 nm is eye-safe, as it would not be able to reach the lens.

### OBJECTIVE OF THE INVENTION

The object of the invention is to provide techniques for producing a UVC laser device in a compact manner, with large conversion efficiency from lasers in the visible spectrum.

### SUMMARY OF THE INVENTION

The object of the invention is achieved by a laser source device. The laser source device includes a pump laser system, a combinator, and nonlinear frequency generation module. The pump laser system is made of one or more laser diodes, each of these laser diodes generates one or more light beams, whose wavelengths are in the visible spectral range which has wavelength in the range 380 nm to 740 nm. The combinator allows passing of the light beams from the pump laser system, and combines them and couple them to the next module. The nonlinear frequency generation module allows passing of the light beams, and while passing through, employs nonlinear optical phenomenon of sum-frequency generation for providing a UVC light beam.

According to another embodiment of the laser source device, the device includes a tunable mechanism which at least tune wavelength of the light beams generated by the laser diode systems, or to tune the phase-matching condition in the nonlinear module. The helps to tune the wavelength of the UVC light beam generated by the device.

According to another embodiment of the laser source device, wherein the tunable mechanism comprises a temperature controller which control temperature of at least one or both of the laser diode systems, or the combinator, or combination thereof. This embodiment provides a simple implementation of tuning by employing temperature change mechanism.

According to yet another embodiment of the laser source device, wherein the light beams generated by each of the laser diode is of different wavelengths. This provides another mechanism for varying the wavelength of the UVC beam generated.

According to one embodiment of the laser source device, the device includes a laser beam generation controller which controls each of the laser diode of the pump laser system for at least varying the intensity of the laser beams or enabling or disabling the laser diodes, or combination thereof. This type of control mechanism is helpful to control generation of the UVC beam, as by varying the intensity of the laser beam, the intensity of the UVC beam can also be changed. Also, enabling and disabling the laser diodes too the change in intensity or wavelength of the UVC beam can be carried out. Even by disabling all the diodes, generation of the UVC beam can be stopped.

According to another embodiment of the laser source device, wherein the combinator is a photonic integrated circuit (PIC). This allows to reduce the number of micro-optical components (lenses, beam splitter, beam expanders, etc) in the system, and optimize the coupling of light between the different chips (the pump lasers and the nonlinear crystal).

According to yet another embodiment of the laser source device, wherein the nonlinear frequency generation module comprises a second-order nonlinear crystal. This provides for simple implementation of the nonlinear frequency generation module's functionality, which is efficient in generating the UVC beams from the laser beams.

According to one embodiment of the laser source device, wherein the second-order nonlinear crystal is a material transparent in at least part of the UVC and part of the visible spectrum. This further enhances the efficiency in generating the UVC beams from the laser beams.

According to another embodiment of the laser source device, wherein the second-order nonlinear crystal is from a selection of barium borate (BBO), cesium lithium borate (CLBO), lithium borate (LBO), potassium dideuterium phosphate (KDP), potassium dideuterium phosphate (DKDP), ammonium dihydrogen phosphate (ADP), yttrium calcium oxoborate (YCOB), or potassium fluoroboratoberyllate (KBBF). This provides for efficient second-order nonlinear crystal which effectively generates the UVC beam from the laser beams.

According to yet another embodiment of the laser source device, wherein the second-order nonlinear crystal is having a geometry of a rib waveguide, defined by a thickness, a width and a depth. This provides for another implementation of the second-order nonlinear crystal in the device.

According to one implementation of the laser source device, wherein the depth is zero. This provides for another geometry of the waveguide commonly known as slab waveguide.

According to another implementation of the laser source device, wherein the depth is equal to the total waveguide thickness. This provides for another geometry of the waveguide commonly known as ridge waveguide.

According yet another embodiment of the laser source device, wherein the nonlinear frequency generation module further comprises a substrate having a material with lower refractive index with respect to the second-order nonlinear crystal, and the second-order nonlinear crystal is coupled to the substrate via wafer bonding.

According to one embodiment of the laser source device, wherein the substrate is made of UV-fused silica.

According to another embodiment of the laser source device, the nonlinear waveguide includes a cladding material having a material with lower refractive index with respect to the second-order nonlinear crystal, which surrounds the second-order nonlinear crystal partially or completely.

According to yet another embodiment of the laser source device, wherein the cladding material is made of calcium fluoride, magnesium fluoride, or a similar material.

According to one embodiment of the laser source device, wherein the UVC light beam is in the range of wavelength between 190 nm to 240 nm.

### BRIEF DESCRIPTION OF DRAWINGS

FIG. 1 illustrates a schematic representation of a laser source device according to a first embodiment of the invention.

FIG. 2 illustrates a schematic diagram of another laser source device according to a second embodiment of the invention, which do not have a top cladding material.

FIG. 3 illustrates a representation of a waveguide which can be used as part of a laser source device according to first embodiment of the invention.

FIG. 4 illustrates a representation of another waveguide which can be used as part of a laser source device according to second embodiment of the invention.

FIG. 5 illustrates a representation of yet another waveguide which can be used as part of a laser source device according to an embodiment of the invention

### DETAILED DESCRIPTION

For the purpose of promoting an understanding of the principles of the invention, reference will now be made to the embodiment illustrated in the figures and specific language will be used to describe them. It will nevertheless be understood that no limitation of the scope of the invention is thereby intended. Such alterations and further modifications in the illustrated system, and such further applications of the principles of the invention as would normally occur to those skilled in the art are to be construed as being within the scope of the present invention.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises . . . a” does not, without more constraints, preclude the existence of other, sub-systems, elements, structures, components, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this invention belongs. The system, methods, and examples provided herein are only illustrative and not intended to be limiting.

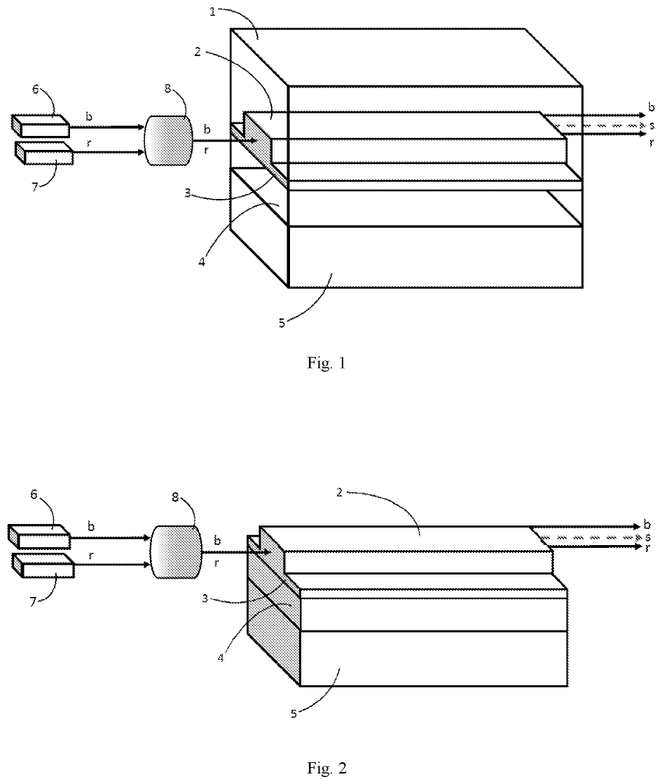

FIG. 1 shows a laser device according to first embodiment of the invention. The laser device includes a pump laser system, a combinator 8, and a nonlinear frequency generation module. The pump laser system shows two laser diode 6, 7 which generates light beams b, r. The light beam's wavelengths are in the visible spectral range which has wavelength in the range 380 nm to 740 nm. The combinator 8 allows further passing of light beams b, r, and while the light beams b, r passes through the combinator 8, it combines the light beams and direct it to the nonlinear frequency generation module. In furtherance, the nonlinear frequency generation module allows passing of the combined light beam b, r through it and while the combined light beam is being passed through, the nonlinear frequency generation module employs nonlinear optical phenomenon of sum-frequency generation for providing UVC light beam s. The UVC light beam s can be in the range of wavelength between 190 nm to 240 nm. While, the combined light beam b, r enters the nonlinear frequency generation module, parts of it gets converted to UVC light beam s, and the remaining non-converted portion of light beam b, r moves out of the nonlinear frequency generation module along with UVC light beam s. In an alternate embodiment, only UVC light beam s leaves out the nonlinear frequency generation module, while the light beam b, r are either reused inside the device allowing for a larger conversion efficiency or blocked.

It is pertinent to be noted that in one embodiment there can be one or more than two laser diodes in the pump laser system.

In one embodiment, the combinator 8 is a photonic integrated circuit (PIC). This PIC can be fabricated with silicon nitride, or aluminum nitride, or available material that is transparent in the relevant wavelength ranges. The PIC facilitates matching of optical modes and guiding them into the nonlinear waveguide.

The nonlinear frequency generation module includes a second-order nonlinear crystal 3. The second-order nonlinear crystal 3 is in a geometry of a waveguide. The second-order nonlinear crystal 3 allows for employing nonlinear optical phenomenon of sum-frequency generation on the combined light beams to generate the UVC light beam.

The second-order nonlinear crystal 3 is made of a material transparent in at least part of the UVC and part of the visible spectrum. In an alternate embodiment, the material of second-order nonlinear crystal 3 is transparent only to the UVC spectrum.

The second-order nonlinear crystal 3 can be made of barium borate (BBO), cesium lithium borate (CLBO), lithium borate (LBO), potassium dideuterium phosphate (KDP), potassium dideuterium phosphate (DKDP), ammonium dihydrogen phosphate (ADP), yttrium calcium oxoborate (YCOB) or potassium fluoroboratoberyllate (KBBF), or combination thereof. All of these crystals are transparent at UVC and will therefore contribute to higher conversion efficiency. Furthermore, all of the mentioned crystals retain second-order susceptibility, allowing them to be used for processes such as second harmonic generation (SHG) and SFG as is seen in the invention.

The nonlinear frequency generation module includes cladding material 1, 4 both on top of the second-order nonlinear crystal 3, and as well as bottom of the second-order nonlinear crystal 3. The cladding material has lower refractive index with respect to the second-order nonlinear crystal 3. The cladding material 1, 4 can be made up of at least one of calcium fluoride, magnesium fluoride, or combination thereof. The cladding material 1, 4 is used to make a larger contrast of refractive index, thus providing a better confinement of the light in the second-order nonlinear crystal 3. In one embodiment, the cladding material 1, 4 may not be provided, or any of the top cladding 1, or the bottom cladding 4 may be provided, however such embodiment shall result in a lesser efficient laser source device due to lack of confinement of the light in the second-order nonlinear crystal 3.

Another advantage of placing cladding material 1, 4 on the top and/or bottom of the second-order nonlinear crystal 3, is that it protects the nonlinear waveguide 3, especially since several of these materials are highly hygroscopic. While the cladding materials used such as calcium fluoride (CaF2) or magnesium fluoride (MgF2) are not hygroscopic, and allows for protecting the nonlinear waveguide 3. Also MgF2 is transparent for wavelengths down to 120 nm.

The nonlinear frequency generation module also includes a substrate 5 having a material with lower refractive index with respect to the second-order nonlinear crystal 3, and the second-order nonlinear crystal 3 is coupled to the substrate 5. The substrate 5 can be made of UV-fused silica. The substrate helps in making a more robust device, both mechanically as well as to protect the second-order nonlinear crystal from it's surrounding environment. In one embodiment, the nonlinear frequency generation module is not provided with the substrate 5.

The second-order nonlinear crystal 3 is defined to a waveguide with specific dimensions to obtain phase matching and good mode overlap. The waveguide geometry allows for light to be guided through total internal reflection by having materials with a smaller refractive index, such as substrate 5 and cladding materials 1, 4 on either side of the second-order nonlinear crystal 3. The waveguide geometry allows for a tight guidance of the light, implying that a smaller effective mode area is achievable, resulting in a stronger electromagnetic intensity in the nonlinear waveguide 3 and thus a larger conversion efficiency compared to bulk crystals. Furthermore, the waveguide geometry allows for phasematching away from the bulk phasematching angle by modal phasematching, thus allowing for a larger conversion efficiency.

The second-order nonlinear crystal 3 has a geometry of a rib waveguide, defined by a thickness 9, a width 12 and a depth 11, as shown in FIG. 4. In this embodiment, the total width 10 of the nonlinear crystal is equal to the width 13 of the entire the nonlinear frequency generation module. In another embodiment, the total width 10 of the nonlinear crystal can be less than the width 13 of the entire the nonlinear frequency generation module, so that the cladding material 1, 4 can cover the nonlinear crystal 3 through out the length 14 of the waveguide 3.

The second-order nonlinear crystal 3 may have any other waveguide geometry, such as slab waveguide, or ridge waveguide. In case of the slab waveguide, the depth 11 is zero. While, in case of ridge waveguide, wherein the depth 11 is equal to the total waveguide thickness 9.

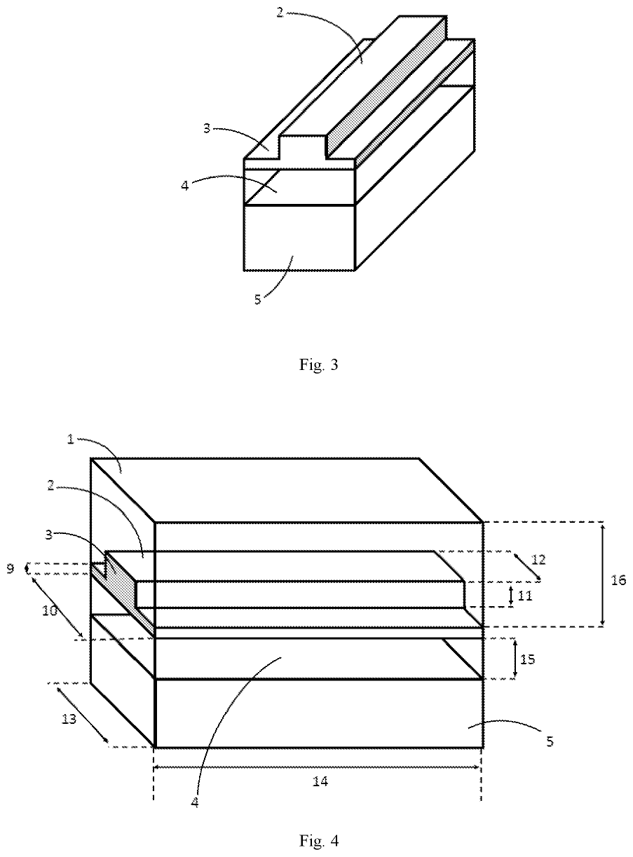

FIG. 2 shows a second embodiment of the laser source device which do not have upper cladding material as part of the nonlinear frequency generation module. All remaining elements are same as the first embodiment in FIG. 1. FIG. 3 shows a front perspective view of the nonlinear frequency generation module of the second embodiment is shown.

FIG. 6 shows schematic of another embodiment of the laser source device 100 which has the tunable mechanism 103 to tune the wavelength of the UVC beam 104. The tunable mechanism can control the pump laser system 101, the nonlinear frequency generation module 102, or the combinator 3. The tunable mechanism 103 controls the pump laser system 101 by tuning the laser diodes to produce wavelength of the light beams for specific wavelengths. The tunable mechanism 103 controls the nonlinear frequency generation module 102 by tuning the phase matching conditions of it. The tunable mechanism 103 controls the combinator 8 to tune the wavelength of the combined laser beams produces by the combinator 8. One way to control the pump laser system 101, the nonlinear frequency generation module 102, or the combinator 3 is based on temperature management of each of these components 8, 101, 102. For this, the tuneable mechanism can have a temperature controller which controls temperature of each of these components 8, 101, 102. By altering the temperature of the pump diode lasers, their wavelengths are shifted. The temperature of the second-order nonlinear crystal can also be changed to tune the phase matching condition. In one embodiment, the tuneable mechanism can only control either one or two of these components 8, 101, 102. The laser source device 100 also includes a laser beam generation controller 105 which controls each of the laser diode of the pump laser system 101 for at least varying the intensity of the laser beams b, r or enabling or disabling the laser diodes, or combination thereof. This helps to vary the wavelengths of the laser beams b, r such that the laser beams b, r can be of different wavelengths.

The shifting of wavelengths of the UVC light beams allow the present invention to be used for spectroscopy, and also to solve problems where light of a specific wave-length in the UVC is essential.

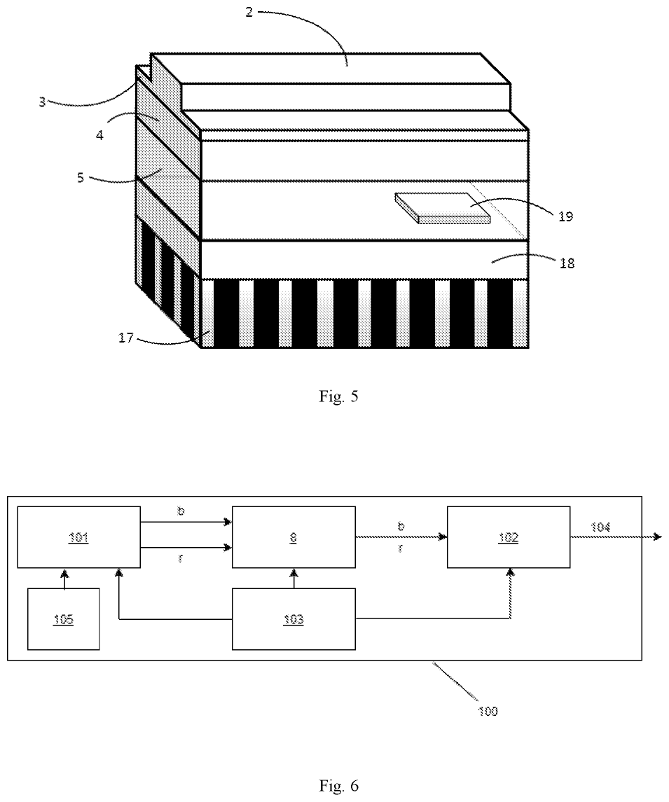

FIG. 5 shows one such tuneable mechanism provided in the nonlinear frequency generation module which includes thermo-electric cooling element 17, heat conducting metallic submount 18, and thermistor 19. The thermistor 19 helps in reading the temperature of the nonlinear frequency generation module, and in furtherance when the temperature reaches above a threshold, a temperature controller gets triggered to control the temperature of the nonlinear frequency module by using the heat conducting metallic submount 18 and the thermo-electric cooling (TEC) element 17, which helps in dissipating heat from the nonlinear frequency module.

It is to be noted that even though this invention is dedicated for germicidal sterilization, but may be used for medical diagnostics, wastewater treatment, gas sensing, telecommunication, UV curing, and many others.

### LIST OF REFERENCE NUMERALS

1. Top cladding material
2. Ridge
3. Nonlinear waveguide slab/Second-order nonlinear crystal
4. Bottom cladding material
5. Substrate
6. First diode laser pump
7. Second diode laser pump
8. Combinator/Photonic integrated circuit (PIC)
9. Thickness/height of the nonlinear crystal
10. Total width of the nonlinear crystal
11. Depth of nonlinear crystal
12. Width
13. Width of the entire the nonlinear frequency generation module
14. Length of the nonlinear crystal/waveguide
15. Height of bottom cladding
16. Height of top cladding
17. Thermo-electric cooling (IEC) element
18. Heat conducting metallic submount
19. Thermistor
b, r Light beams
100 Laser source device
101 Pump laser system
102 Non-linear frequency module
103 Tunable mechanism
104 UVC Beam
105 Laser beam generation controller

1. A laser source device comprising:
a pump laser system, made of one of more laser diodes, each of these laser diodes is adapted to generate one or more light beams, whose wavelengths are in the visible spectral range having a wavelength in the range 380 nm to 740 nm;
a combinator adapted to allow passing of the light beams from the pump laser system, and to combine them and couple them to a next module;
a nonlinear frequency generation module adapted to allow passing of the light beams, and while passing through, adapted to employ nonlinear optical phenomenon of sum-frequency generation for a UVC light beam.

2. The laser source device according to the claim 1 comprising:
a tunable mechanism adapted to at least tune wavelength of the light beams generated by the pump laser system, or to tune the phase-matching condition in the nonlinear frequency generation module.

3. The laser source device according to the claim 1, wherein the tuneable mechanism comprises a temperature controller adapted to control temperature of at least one or both of the pump laser system, or the combinator, or combination thereof.

4. The laser source device according to claim 1, wherein the light beams generated by each of the laser diode is of different wavelengths.

5. The laser source device according to the claim 4 comprising a laser beam generation controller adapted to control each of the laser diode of the pump laser system for at least varying the intensity of the laser beams or enabling or disabling the laser diodes, or combination thereof.

6. The laser source device according to claim 1, wherein the combinator is a photonic integrated circuit (PIC).

7. The laser source device according to claim 1, wherein the nonlinear frequency generation module comprises a second-order nonlinear crystal.

8. The laser source device according to the claim 7, wherein the second-order nonlinear crystal is a material transparent in at least part of the UVC and part of the visible spectrum.

9. The laser diode device according to claim 8, wherein the second-order nonlinear crystal is from a selection of barium borate (BBO), cesium lithium borate (CLBO), lithium borate (LBO), potassium dideuterium phosphate (KDP), potassium dideuterium phosphate (DKDP), ammonium dihydrogen phosphate (ADP), yttrium calcium oxoborate (YCOB) or potassium fluoroboratoberyllate (KBBF).

10. The laser source device according to claim 7, wherein the second-order nonlinear crystal is having a geometry of a rib waveguide, defined by a thickness, a width and a depth.

11. The laser source device according to the claim 10, wherein the depth is zero.

12. The laser source device according to claim 10, wherein the depth is equal to the total waveguide thickness.

13. The laser source device according to claim 7, wherein the nonlinear frequency generation module further comprises a substrate having a material with lower refractive index with respect to the second-order nonlinear crystal, and the second-order nonlinear crystal is coupled to the substrate.

14. The laser source device according to the claim 13, wherein the substrate is made of UV-fused silica.

15. The laser source device according to claim 7, wherein the nonlinear frequency generation module further comprises a cladding material having a material with lower refractive index with respect to the second-order nonlinear crystal, which surrounds the second-order nonlinear crystal partially or completely.

16. The laser source device according to the claim 15, wherein the cladding material is made of at least one of calcium fluoride, magnesium fluoride, or combination thereof.

17. The laser source device according to claim 1, wherein the UVC light beam is in the range of wavelength between 190 nm to 240 nm.
